(19) United States
(12) Patent Application Publication
(10) Pub. No.: US 2007/0298060 A1
(43) Pub. Date: Dec. 27, 2007

(54) METHOD FOR DEPLETION OF CARIES-CAUSING BACTERIA IN THE ORAL CAVITY

(75) Inventors: Steven D. Goodman, Redondo Beach, CA (US); Jennifer S. Downey, Simi Valley, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 11/760,626

(22) Filed: Jun. 8, 2007

Related U.S. Application Data
(60) Provisional application No. 60/812,240, filed on Jun. 9, 2006.

(51) Int. Cl. A61K 8/02; A47L 23/02; A61K 9/68; A61P 1/02; A61K 8/00

(52) U.S. Cl. 424/401; 15/28; 424/440; 424/49

(57) ABSTRACT

Herein is provided a method for treating or preventing caries by applying a caries-causing agent removal device, a device capable of selectively and preferentially binding the caries-causing agent compare with other surrounding non-cariogenic organisms. There is also provided a set of compounds for formulating the device thereof. Exemplary compounds include glycosidic polymers such as Sephadex®. Specific examples of a device of the present invention include candy, chewing gums, mouthwash, and toothpaste

### CROSS-REFERENCE TO RELATED APPLICATIONS

This application claims an invention which was disclosed in Provisional Application No. 60/812,240, filed Jun. 9, 2006, entitled “A METHOD FOR DEPLETION OF CARIES-CAUSING BACTERIA IN THE ORAL CAVITY”. The benefit under 35 USC §119(e) of the U.S. provisional application is hereby claimed. The above priority applications are hereby incorporated herein by reference.

### STATEMENT REGARDING FEDERALLY SPONSORED RESEARCH AND DEVELOPMENT

The present invention is made, at least in part, with the support of NIH grant R01 DE13965. The government has certain rights in the invention.

### FIELD OF THE INVENTION

The present invention generally relates to the field of dental care. More particularly, the present invention pertains to a method for removing caries-causing bacteria from the mouth. The present invention also pertains to products and compounds for removing caries-causing bacteria from the mouth.

### BACKGROUND OF THE INVENTION

Dental caries is a transmissible infectious disease that is present in 94% of adults with one or more natural teeth. According to the 2000 Surgeon General's report on oral health care in America, 78% of 17 year olds have at least one decayed tooth or filling. In addition, a national survey from 1988-1994 showed that about 18% of US teens have untreated dental decay and about 4% have decayed, missing and filled surfaces in permanent teeth. The Surgeon General reports that the cost of oral health care alone was responsible for 4.7% of the nation's health expenditures in 1998, costing the nation approximately $53.8 billion. Therefore researchers are continually investigating possible new avenues for the prevention of tooth decay.

Dental caries is described as the demineralization of the tooth surface caused by bacteria. Localized breakdown of the tooth enamel is caused by acids (particularly lactic acid) produced by plaque microorganisms that are able to bind to the surface of the tooth. Dental plaque accumulates on the tooth surface and is comprised of a large and diverse population of microbes representing over 700 bacterial species of which only 50% are able to be cultivated. Among these oral bacteria Streptococcus mutans (“S. mutans”) has been shown to be the major contributor to dental caries. S. mutans converts foods, especially sugars and starch, into acid which can seep into the tooth and breakdown the enamel. A correlation has been shown between the presence of S. mutans in patients with caries and the absence of S. mutans in the mouth of patients that lack dental caries. In addition, the amount of S. mutans present in the mouth has been shown to correlate with the individual's dietary intake of sucrose.

Plaque bacteria begin to accumulate on teeth within 20 minutes of eating. If not removed thoroughly and routinely, the bacteria will flourish and tooth decay will begin. The standard methods for prevention of tooth decay include brushing the teeth for two minutes twice daily, flossing at least once a day, regular dental checkups and limiting dietary intake of sugar.

Additional methods of prevention include dental sealants, fluoride, chlorhexidine, salivary enhancers and antimicrobial agents. Dental sealants are thin plastic-like coatings that are applied to the chewing surfaces of the molars to prevent the accumulation of plaque in the pits and fissures of the tooth. Individuals drinking water with fluoride or taking fluoride supplements have been shown to develop fewer dental caries. In children, fluoride ingested while teeth are developing is incorporated into the tooth enamel to help fight the destruction from acids. In addition, most toothpaste includes a topical fluoride to apply directly to the tooth. Fluoride in conjunction with calcium, phosphate prevents the loss of minerals from the tooth surface, enhances the uptake of minerals to aid in the remineralization of the tooth, inhibits bacterial production of acids and, at high concentrations, can inhibit bacterial growth.

Chlorhexidine is an antiseptic that works as a broad-spectrum antibacterial agent. The exact mode of action is not known but chlorhexidine is believed to affect cell membrane function (decreased permeability). Twice daily rinses with chlorhexidine inhibit plaque formation and prevent gingivitis by killing all oral bacteria, however undesirable side effects are encountered (spotting on the teeth and altered ability to taste). Saliva is also an important cleansing agent to help remove food debris from the mouth. Saliva is rich in calcium that acts as a buffer by helping neutralize acid. Salivary enhancers and drinking plenty of water can help remove food debris and prevent tooth decay. In addition antimicrobial decapeptides have been used for the prevention of tooth decay by adding them to chewing gum as an antiplaque agent.

Chewing gum has become a popular mode of delivery for various drugs to help prevent dental caries as well as a method to deliver drug therapy for treatment of other maladies. For example, non-medicated sugar free chewing gum containing a sugar substitute, xylitol, is recommended to patients with severe dry mouth to help stimulate the release of saliva and in children to reduce dental caries. Carbamide, bicarbonate, fluoride, chlorhexidine and various enzymes have also been added to chewing gum to help prevent dental caries. In addition, chewing gum has been utilized as a drug delivery system for oropharyngeal infections, post-operative care of tonsillectomised patients, delivery of drugs to aid in smoking cessation, and to patients recovering from narcotic addiction.

The advantage of using chewing gum as a drug delivery system is that it can be taken without water and can be administered discreetly. Released drugs can treat oral diseases locally or can be absorbed through the oral mucosa for a systemic effect. However if the drug is not soluble it may not be released from the chewing gum at the correct time, or in the right dose.

Recently, investigators from the University of Kentucky, in conjunction with the United States Army, have started developing chewing gum containing an antimicrobial decapeptide (KSL) to help prevent dental caries in soldiers in the field where the opportunity for oral healthcare is not optimal. This novel decapeptide is reported to have a broad range of antibacterial activity by interacting directly with microbial surfaces and disrupting membrane permeability. However, this method also suffers similar drawback as other drugs in that it must be released from the chewing gum, diffuse to the location of the bacteria, and penetrate the biofilm layer to come in contact with the bacteria. Because KSL is a broad spectrum antibacterial agent, it also suffers from the problem of being non-specific.

All of the above prevention techniques aid in the decrease of tooth decay. However, most are general methods of prevention and there is no method available that is specific for the bacteria that cause dental caries and to the areas where the treatment is needed. One goal of the present invention is to develop a method that specifically targets S. mutans, the primary causative agent of dental caries and will remove the bacteria from the oral cavity without adversely affecting the other non-pathogenic bacteria of the oral cavity or the human host.

S. mutans is a Gram type-positive facultative anaerobe that is commonly found in the oral cavity and is recognized as one of the principal bacteria associated with tooth decay. The sequence of the genome of S. mutans strain UA159, released in 2002, revealed a circular chromosome with an average GC content of 36.82%. It has 63% of predicted open reading frames (ORFs) with an assigned function, 21% are homologous with different species and 16% are unique to S. mutans. Of those ORFs, known virulence genes associated with putative hemolysins, acid tolerance, proteases, adhesion, and extracellular glucan production were identified, in addition to genes necessary for natural competency and quorum sensing. In order for S. mutans to survive and propagate in a host its ability to adhere to the host's tooth and grow on the surface of the tooth is essential. This is reflected in the fact that edentate hosts fail to harbor S. mutans. However, to stick to a tooth surface, S. mutans must alter gene expression to transform from a free-living, planktonic form, to a biofilm form attached to the surface of the tooth.

Biofilm formation by S. mutans can occur by two methods. In the first method, S. mutans attachment is mediated by several surface adhesion proteins (such as streptococcal protein antigen P, SpaP), and glucan binding proteins A, B, C & D (GbpA, GbpB, GbpC & GbpD). These proteins mediate the attachment to salivary agglutinin as well as to other bacteria and extracellular matrixes. In the second method of attachment, S. mutans uses glucosyltransferases (GTFs) to synthesize glucans from sucrose. The glucans in turn mediate the efficient attachment of the bacteria to the tooth surface, or promotes cell-cell aggregation.

Evidence shows that 65% of human bacterial infections involve a critical biofilm phase. Biofilms are typically comprised of a complex mixed population of multiple bacterial species, and form upon binding to a surface into communities with division of labor, intercellular communication and intricate structures embedded in an exopolymeric matrix (e.g. exopolysaccharide and nucleic acid). The exopolysaccharide (EPS) layer helps to protect the bacteria from the various stresses, e.g. dental biofilms or dental plaque can encounter nutrient shortage or excess, low pH, high osmolarity, and consumption of antimicrobial agents or antibiotics by the host.

As mentioned above, dental plaque is comprised of a large and diverse population of microbes representing over 700 species. A large portion of the oral bacteria are naturally occurring non-pathogenic commensal bacteria that play important roles in human health. These bacteria can help out compete and prevent colonization by pathogens and stimulate immune functions. As with any part of the human body a disruption of the natural flora can lead to diseases, which, in the oral cavity include dental caries and periodontal disease. Maintenance of a healthy commensal flora in the mouth is also believed to discourage other pathogenic bacteria and yeast from transiently colonizing the mouth where they can then disseminate to other areas of the body and cause disease. Thus, there is an increased need to develop methods to treat the bacteria that are causing the disease without disturbing the commensal bacteria.

Currently, all prior art prevention methods as exemplified by the methods described above are broad-spectrum and do not distinguish between the different bacterial species found in the oral cavity. Some agents may even be toxic (e.g. fluoride and chlorhexidine) and can only be applied by dental professionals. Antibacterial agents often have undesirable taste (e.g. chlorhexidine) and may stain teeth and mucosal tissues in the mouth. Moreover, current delivery methods require that these agents be released from the product to reach the colonized area of the oral cavity, which presents an additional challenge that may reduce the effectiveness of the agent.

Therefore, there still exists a need for a method of selectively and effectively removing caries-causing bacteria from the oral cavity that is also easy to administer.

### SUMMARY OF THE INVENTION

In view of the above, it is one object of the present invention to provide a method for selectively removing caries-causing bacteria, specifically S. mutans, from the oral cavity that is also easy to administer.

In accordance with this object, a family of well-characterized non-toxic compounds that are insoluble is identified in the present invention. One advantageous feature of these new compounds is that they will remain out of solution, thereby, achieving a prolonged action when included in chewing gum, toothpaste or suspended in mouthwash. These compounds do not have antimicrobial properties but rather bind specifically to S. mutans. Once the bacteria bind to the compound both can be expelled (spit) out of the mouth or even swallowed, thus physically removing the bacteria that cause dental caries. The complete binding occurs in less than a minute and is selective for S. mutans. Hence the more frequent the use of the compound the smaller the reservoir of the caries-causing bacteria in the oral cavity. A therapeutic oral healthcare line to specifically remove or reduce plaque-causing bacteria from the oral cavity without disturbing the healthy commensal bacteria population is developed accordingly.

Other aspects and advantages of the invention will be apparent from the following description and the appended claims.

### BRIEF DESCRIPTION OF THE DRAWINGS

FIG. 1 illustrates S. mutans binds to Sephadex® G-25 and the assay removes the bound S. mutans from the flow through. S. mutans cultures were grown to an OD_{650 nm} 0.07 as planktonic (P) cultures with sucrose (S) and without (−) or with (+) Sephadex®. Percent unbound cells were calculated separately for each attempt by setting without Sephadex® as 100% and with Sephadex® calculated compared to without Sephadex®.

FIG. 2 shows incubation of S. mutans UA159 with 10% Sephadex® G-25 for 0-30 minutes. S. mutans cultures were grown to an OD_{650 nm} 0.3 or 0.6 as planktonic (P) cultures with (S) and without sucrose and without (−) and with (+) Sephadex® for 0, 1, 5, 10 and 30 minutes. Percent unbound cells are calculated separately for each condition without Sephadex® set as 100% and with Sephadex® calculated compared to without Sephadex®. (A) OD_{650 nm} 0.3 no sucrose (B) OD_{650 nm} 0.6 with sucrose.

FIG. 3 shows incubation of A) S. gordonii and B) E. coli with 10% Sephadex® G-25 for 1 minute. S. gordonii and E. coli cultures were grown to OD_{650 nm} 0.3 or 0.6 as planktonic (P) cultures with (S) and without sucrose and without (−) and with (+) Sephadex® for 1 minute. % unbound cells were calculated separately for each condition setting without Sephadex® as 100% and with Sephadex® calculated compared to without Sephadex®.

FIG. 4 shows incubation of S. mutans with A) 2% Sephadex® G-100 or B) 4% G-50 Sephadex® for 1 minute. S. mutans UA159 cultures were grown to OD_{650 nm} 0.3 or 0.6 as planktonic (P) cultures with (S) and without sucrose and without (−) and with (+) Sephadex® for 1 minute. Percent unbound cells were calculated separately for each condition setting without Sephadex® as 100% and with Sephadex® calculated compared to without Sephadex®.

FIG. 5 shows incubation of S. mutans with 10% Sephadex® G-25 slurry for 1 minute in THB versus artificial saliva. S. mutans UA159 cultures were grown to OD_{650 nm} 0.3 under planktonic (P) conditions in THB. 1 ml of culture was harvested by centrifugation (380×g, 1 minute) and resuspended in 1 ml THB or artificial saliva. 0.5 ml of culture in THB or artificial saliva was incubated with 0.2 ml 10% Sephadex® G-25 for 1 minute. Percent unbound cells were calculated separately for each condition, setting without Sephadex® as 100% and with Sephadex® calculated compared to without Sephadex®.

FIG. 6 shows incubation of S. mutans with 10% Sephadex® G-25 slurry for 1 minute in THB with 0-10% SDS, S. mutans UA159 cultures were grown to OD_{650 nm} 0.3 under planktonic (P) conditions in THB. 0.2 ml culture was incubated with 0.2 ml 10% Sephadex® G-25 in THB containing 0-10% SDS for 1 minute. Percent unbound cells were calculated separately for each condition, setting without Sephadex® as 100% and with Sephadex® calculated compared to without Sephadex®.

FIG. 7 shows the result of competition assay in which S. mutans cultures are in the presence of both a hydroxyapatite (HA) disc and Sephadex® for a short period of time (5 minutes). +/H culture=cells remaining in supernatant after exposure to Sephadex®; +/H HA=cells bound to HA disc; +/HS culture=cells remaining in supernatant after exposure to HA disc & Sephadex®; +/HS seph=cells bound to Sephadex®; +/HS HA=cells bound to HA disc after exposure to HA disc & Sephadex®.

FIG. 8 shows percentage of binding of various S. mutans wild type strains grown as planktonic cultures in the absence or presence of 3% sucrose. ND=Not Determined.

FIG. 9 shows percentage of binding of non-Streptococcus strains in the absence or presence of 3% sucrose. ND=Not Determined.

FIG. 10 shows percentage of binding of various Streptococcus species grown in a biofilm in the absence or presence of 3% sucrose.

FIG. 11 shows the result of Sephadex® pulling S. mutans out of a 19.5 hour biofilm ±2′vortex=S. mutans released from a biofilm into the culture supernatant from a HA disc without incubation with Sephadex® after 2 minutes of vortexing; +/+2′vortex=S. mutans released from a biofilm into the culture supernatant from a HA disc with incubation with Sephadex® after 2 minutes of vortexing.

FIG. 12 shows the result of binding of different S. mutans strains to Sephadex® in 16-20 hour biofilm.

FIG. 13 shows percentage of binding of planktonic cultures of various Streptococcus species.

### DETAILED DESCRIPTION

As outlined above, the present invention provides an alternative method that overcomes these difficulties of the above mentioned methods. Specifically, the present invention provides a more specific way to remove the caries-causing bacteria from the oral cavity by specifically removing S. mutans from the oral cavity without significantly affecting the commensal bacteria. Although not intended to be limited, a brief discussion of the process by which S. mutans bind to the surface of the tooth will facilitate a more complete understanding of the present invention for the reader.

Binding of S. mutans to the surface of a tooth is achieved only through attachment of the biofilm. Thus, in order for S. mutans, to bind to a tooth, it must alter its gene expression to transition from planktonic cells to a biofilms state. This attachment event can generally occur in two ways.

In the first scenario, attachment of S. mutans utilizes sucrose as a substrate to transfer a glucose moiety to a growing polysaccharide chain of glucose subunits, referred to as glucans. One of the major virulence properties of S. mutans are GTFs that synthesize glucans de novo from sucrose, which then mediate the attachment to the tooth surface and promote cell-cell aggregation. S. mutans contains three GTFs (GtfB, GtfC, GtfD), all of which are highly homologous (at least 50%), while GtfB and GtfC are more than 75% identical and arranged in direct repeat within the chromosome.

Although the GTFs are highly homologous, the glycosidic linkages of the glucan products distinguish their activities. GtfB produces primarily mutan, a water-insoluble α-1-3 glycosidic linked glucan, GtfD produces primarily dextran, a water-soluble α-1-6 glycosidic linkage, whereas, GtfC generates both products. Mutational analyses of S. mutans demonstrated that in in vitro assays in the presence of sucrose, water-insoluble glucans (derived from GtfB and GtfC) play an essential role in adherence by facilitating the attachment of bacteria to the tooth pellicle and promoting plaque biofilm formation. In contrast, GtfD was observed to have an important role in the structure of the biofilm and may act as an extracellular storage polymer that is degraded to metabolize carbohydrates during periods of low carbohydrate availability and promote cell-cell aggregation.

In the second scenario, S. mutans attachment is mediated by several surface adhesion proteins, which mediate attachment to salivary agglutinin, other bacteria, and extracellular matrixes. It has been shown that Streptococcus sobrinus and S. mutans exhibit rapid aggregation of cells under stress conditions. This observation is defined as dextran (α-1,6 glucan)-dependent aggregation (ddag). To induce the ddag phenomenon cells were grown under stress induced by antibiotics (including tetracycline or spectinomycin), amino acid analogs, ethanol, xylitol or elevated temperatures. An extracellular protein, glucan binding protein C (GbpC), was identified by mutagenesis studies as the protein responsible for this binding. GbpC attachment to dextran occurs under laboratory induced stress conditions and during initial binding to the tooth.

Accordingly, in one aspect, the present invention provides a method for selectively removing caries-causing agents from a site. Methods according to embodiments of the present invention generally comprises the steps of applying a glucosidic polymer-based caries-causing agent removal device to the oral cavity so as to cause attachment of the caries-causing agents to the device; and removing the device from the oral cavity, thereby, removing the caries-causing agents from the oral cavity.

While the human oral cavity is contemplated as the preferred site for application of a device of the present invention, this is not required. The site may also be the oral cavity of an animal such a dog, a horse, a cat, or any other animals that have oral cavity and teeth.

Generally speaking, any glucosidic polymer having α-1,6 linkage, α-1,3 linkage, or any combinations thereof may be advantageously employed. The device generally takes the form of an elastic solid body. Preferably, the main material of the body is a dextran-based polymer. More preferably, it is a Sephadex®, a mimetics thereof, or a combination thereof. It is also preferably insoluble in water or saliva.

The dextran-based caries-causing agent removal device may be any suitable formulation of dextran. In one preferred embodiment, an insoluble spherical cross-linked dextran, Sephadex® (GE Healthcare, Pistcataway, N.J.) is used. Sephadex® is typically used for size exclusion chromatography in the laboratory setting.

It is an unexpected discovery of the present invention that the major caries-causing agent, S. mutans, readily stick to Sephadex® as a whole cell. Thus, by contacting S. mutans to a Sephadex®-based removal device, S. mutans will preferentially bind to the removal device and be physically removed from the oral cavity. Other species of Streptococci may also be selectively removed from an environment/site that has non-Streptococci species of microbes.

Exemplary species of Streptococci that bind to Sephadex® or sepharose; S. mutans, S. sobrinus, S. oralis, S. sanguis, S. gordonii, S. mitis, S. salivarius, S. cristatus or a combination thereof.

Other formulations of dextran-based caries-causing agent removal device may further include other ingredients such as artificial flavors, antibiotics, a dextran-dependent aggregation inducer, or any other suitable oral hygiene enhancer commonly known in the art.

The amount of S. mutans in human saliva can vary between 0-10⁷ colony forming units/ml depending on the individual. To account for this individual variation, various phases of cell growth (lag, mid-log and early stationary phase) of culture should be used to determine the appropriate dosing information for a particular formulation of Sephadex®-based S. mutans removal device.

The device can be realized in a number of different dental care products. Thus, in another aspect, the present invention also provides dental care products and tools for the prevention or treatment of caries as well as the removal of caries causing agents. In one preferred embodiment, the device is realized in the form of chewing gums. Other exemplary embodiments may include, but not limited to candy, mouthwash, toothpaste, or a combination thereof.

As described above, methods of the present invention generally comprises the steps of applying the device to the oral cavity so as to cause attachment of the undesirable agents to the device, and then removing the device from the oral cavity to dispose of the undesirable agents. In further embodiments of the present invention, the method may farther include timing considerations, frequency, and combined use with other dental care products. Depending on the particular product form of the removal device, the manner in which the removal device is disposed of may also vary. This fact will be readily appreciated by a person skilled in the relevant art.

For example, an exemplary method of the present invention may comprise applying a dextran-based chewing gum to the oral cavity wherein the manner of application is chewing and wherein the duration of chewing may vary from a few seconds to a few minutes. Moreover, the chewing gum may be disposed of either by swallowing or by disposing it at an external location such as a trash can. Based on the above description, other variations will be apparent to one skilled in the relevant art.

There are several advantages for using dextran to formulate a caries-causing agent removal device of the present invention. Dextran has been shown to have beneficial activity for medical treatments. Dextran is given intravenously for its anti-platelet activity, anti-fibrin activity and improving microcirculation by decreasing blood viscosity and impeding erythrocyte aggregation. In addition microsurgeons use dextran to decrease clot formation by binding platelets, red blood cells, and vascular endothelium decreasing their ability to stick together. Dextran is also used in some eye drops as a lubricant and in certain intravenous fluids to solubilize other factors, e.g. iron-dextran. Complications and side effects from the use of dextran are rare but can include vomiting, fever, joint pains, rash, tightness in chest area and swelling.

Experiments in rats show limited metabolism of Sephadex®. There is no apparent toxicity and the material data safety sheet for Sephadex® G-25 reports no known potential human health effects. Accordingly, in the present invention, Sephadex® is used as a vehicle to deplete S. mutans from the oral cavity.

To further demonstrate the present invention, the following examples are provided.

### EXAMPLES

Dextran Binding Assay

An assay was developed to bind S. mutans (strain UA159 ) to the Sephadex® and quantify the amount of bacteria that adhered. For this assay, overnight cultures were diluted into fresh Todd Hewitt Broth (THB) with or without 3% sucrose and grown at 37° C. with 5% CO₂ while the OD_{650 nm} was monitored (an OD6_{50 nm} of 0.1 is estimated to be 10⁸ colony forming units/ml of culture).

Once the culture reached an OD_{650 nm} of 0.07, 0.5 ml of culture was added to an empty Micro Bio-Spin chromatography at column (BioRad, Hercules, Calif.) with or without adding an equal volume of a 10% slurry of Sephadex® G-25 hydrated in THB.

Cells and Sephadex® were incubated at room temperature for 1 minute and then unbound cells were harvested by centrifugation at 380×g for 1 minute. To calculate the percentage of unbound cells the OD_{650 nm} of the cells that went through the column were measured. For each condition a parallel reaction without Sephadex® was set to 100% for comparison.

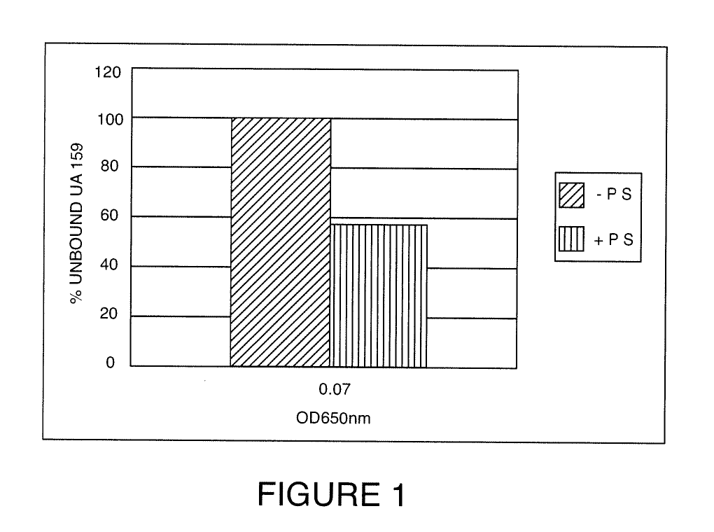

FIG. 1 illustrates the efficiency of binding under the initial conditions and that S. mutans bound to Sephadex® G-25 and about 40% of the cells were removed from the flow through.

Time Course of Binding

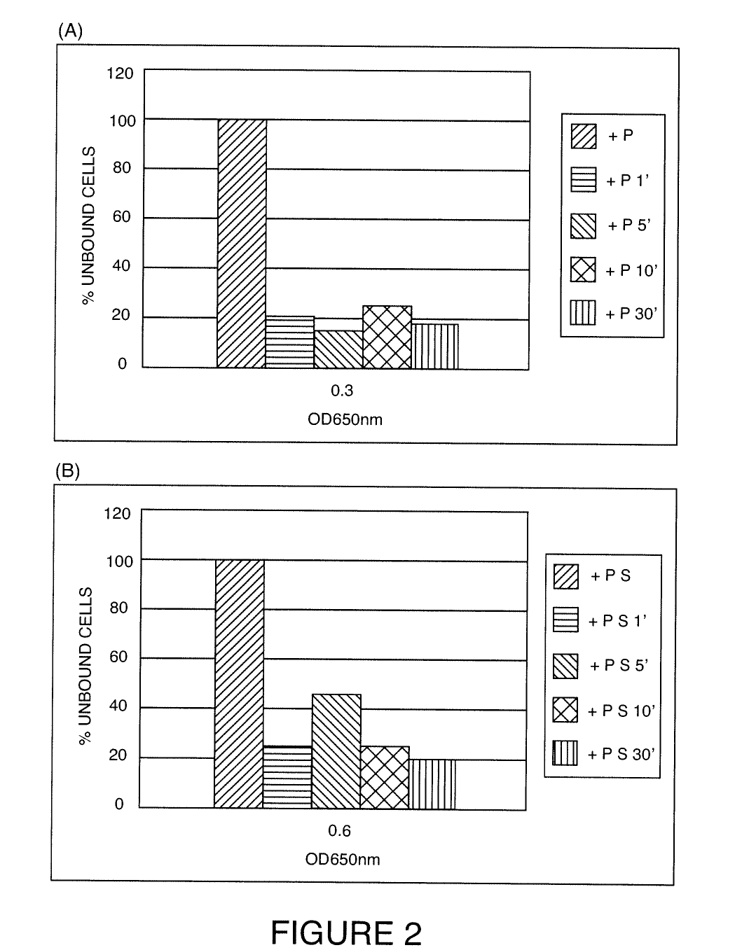

After the efficient binding of S. mutans UA159 to Sephadex® G-25, the incubation time for the bacteria with the Sephadex® was optimized. FIG. 2 shows the results from incubating S. mutans UA159 (OD_{650 nm} 0.3 and 0.6) with Sephadex® for 0 to 30 minutes, and that after one minute most of the binding had already occurred.

Accordingly, Sephadex® in toothpaste, mouthwash or chewing gum has a reasonable window of time to bind S. mutans.

S. mutans Strains Specificity

Many different strains of the S. mutans can be found in the human population at large, although most people have two, a major and a minor species in their oral cavity. To demonstrate that this assay is applicable to other S. mutans strains, three other wild-type S. mutans strains (UA140, NG8 & BM71) were grown to various growth phases, incubated with the slurry of 10% Sephadex® G-25 for one minute and the percentages of unbound cells were calculated as described above. Results for planktonic cultures grown in the presence of sucrose for S. mutans strains UA140, NG8 and BM714 to an OD_{650 nm}, of 0.3 had 32%, 7% and 6% unbound, respectively, and those at an OD_{650 nm} of 0.6 had 31%, 33% and 13% unbound respectively (data not shown), demonstrating that heterogeneity between strains of S. mutans do not interfere with binding to Sephadex®.

S. mutans Selective Binding Over Other Bacteria

For methods of the present invention to be most useful for the treatment of dental caries, the caries-causing bacteria are specifically depleted while leaving the remaining commensal bacteria in the oral cavity. The Escherichia coli DH5 (an intestinal bacteria that needs to pass through the mouth to get to its native site) and Streptococcus gordonii (a commensal resident of the oral cavity and early tooth colonizer) were incubated individually with Sephadex® G-25 for one minute at room temperature.

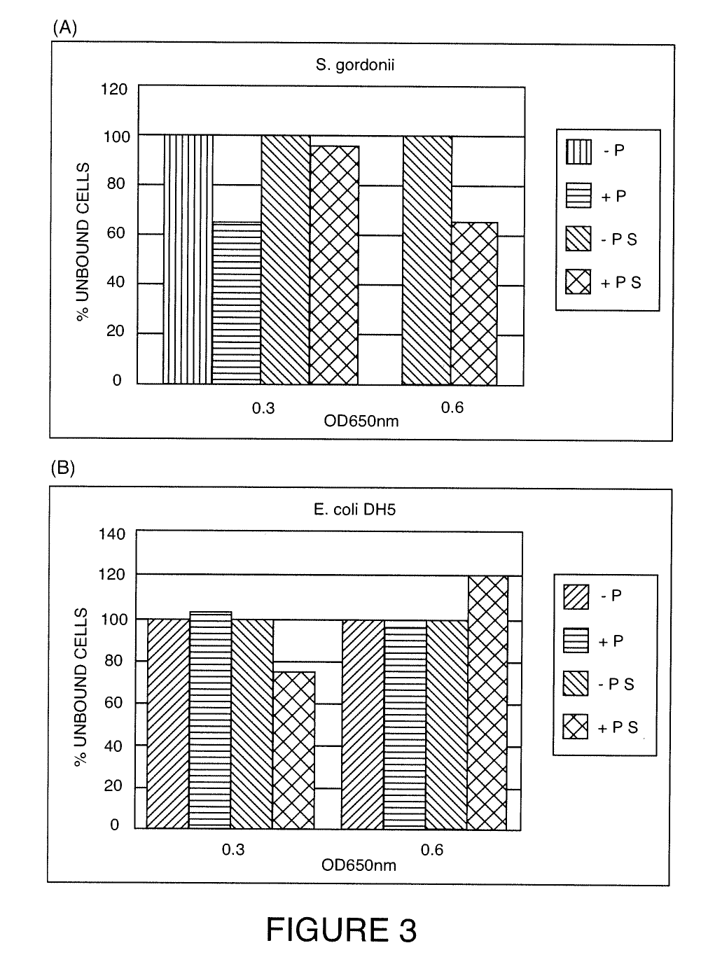

Data from FIG. 3 shows that these bacteria could only achieve minimal binding to Sephadex®, illustrating that other bacteria in the oral cavity are unlikely to be perturbed.

Testing Different Sizes of Sephadex®

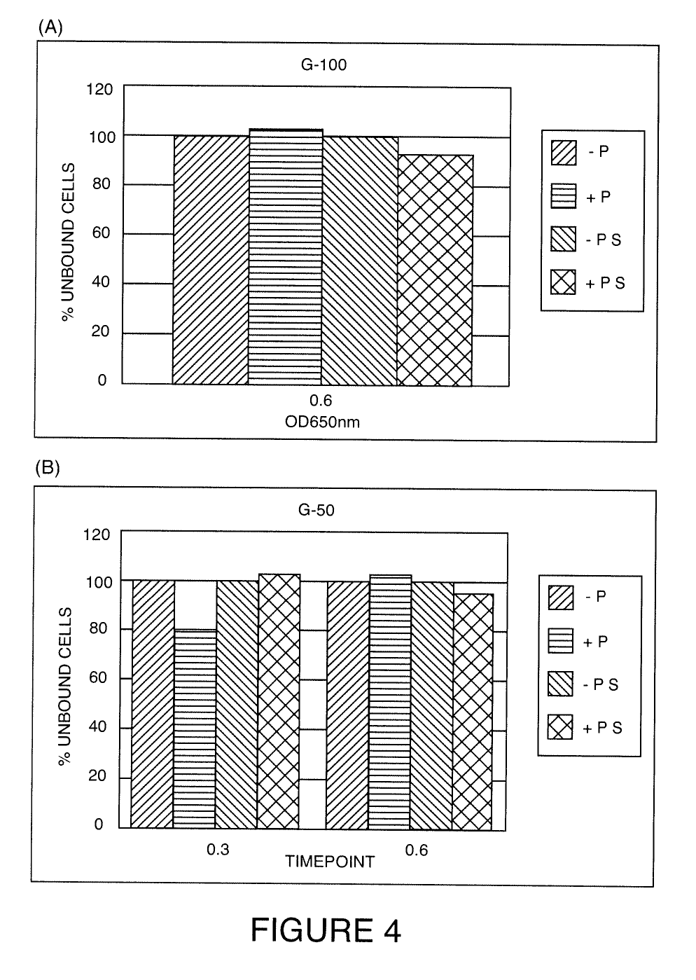

Sephadex® G-25 was first analyzed because of the small particle size (35-140 μM diameter, hydrated). The 11 commercially available Sephadex® for binding to S. mutans were also evaluated. Cultures were grown as previously described and at the appropriate densities, cultures were incubated with 0.15 ml slurry of either 2% G-100 or 4% G-50 Sephadex® hydrated in THB. FIG. 4 shows that in comparison to Sephadex® G-25, Sephadex® G-100 and G-50 do not bind S. mutans as efficiently. Demonstrating that the smaller the beads the higher percentage of S. mutans cells removed and that G-25 or possibly smaller would be the optimal for therapeutic use.

Saturation of S. mutans Binding to Sephadex®

Next, the bacterial binding capacity (saturation) of Sephadex® and what amount of bacteria present in the oral cavity Sephadex® would be capable of removing were determined. In an individual with severe caries the saliva can contain up to 10⁸ cells. S. mutans UA159 planktonic cells were grown to mid log phase (OD_{650 nm} 0.25) and incubated at room temperature on a Micro BioSpin column with 0.5 ml of 10% Sephadex® G-25 slurry (˜50 mg, “pea-sized” amount) for 1 minute, unbound cells were removed by centrifugation (2,000 rpm, 1 minute), then another 0.5 ml of culture was added, centrifuged and this was repeated for up to a total of 10 ml of culture.

Results demonstrated that even after 5.5 ml of an OD_{650 nm} 0.25 S. mutans UA159 culture (˜10⁹ cells) saturation of the Sephadex® was not observed (data not shown). This was repeated with S. mutans grown in the presence and absence of 3% sucrose and incubated with 0.25 ml of 10% Sephadex® G-25 and again no saturation was observed after 10 ml of culture over the column (data not shown). Demonstrating that a minimal amount of Sephadex® is required to remove S. mutans from the oral cavity.

Sticking in Artificial Saliva

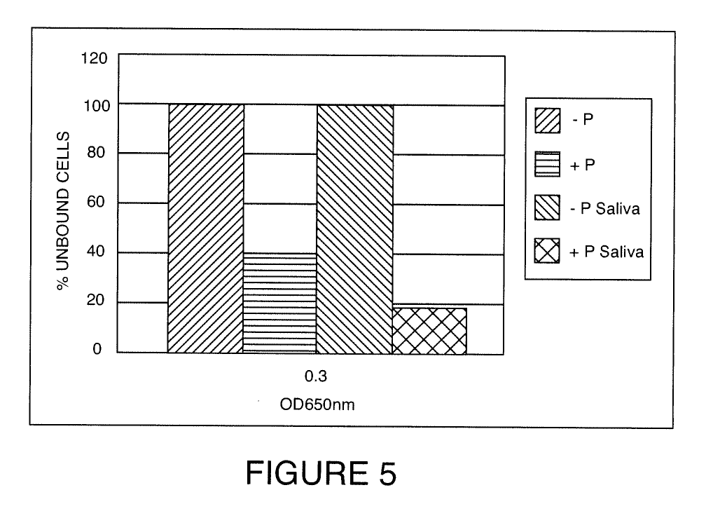

S. mutans is typically grown in THB which is a nutrient rich growth media and all of our assays have thus far been performed in this media. An artificial saliva to assay the solubility of decapeptide KSL over time is utilized. This artificial saliva was composed of 14.49 mM sodium chloride, 16.09 mM potassium chloride, 1.31 mM calcium chloride, 0.54 mM magnesium chloride, and 1.96 mM potassium phosphate diabasic at a final pH of 5.7. As a starting condition to mimic S. mutans binding Sephadex® in the oral cavity, 1 ml of S. mutans culture was harvested by centrifugation and resuspended the cells in 1 ml of artificial saliva. These cells were then incubated with Sephadex® on the column and unbound cells were measured. FIG. 5 demonstrates that the use of artificial saliva compared to THB has no effect of the binding of S. mutans to Sephadex®, suggesting that the reaction should occur in the oral cavity.

Initial Testing of Sephadex® Binding in Oral Healthcare Excipients

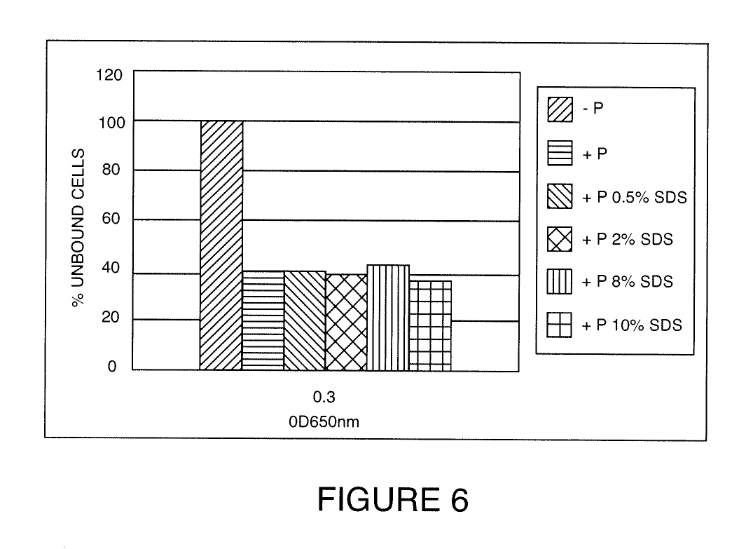

A major component of toothpaste, a detergent, sodium dodecyl sulfate (SDS; typically making up 5 to 8% of dentifrice) and its effect on the binding of S. mutans to Sephadex® was investigated. FIG. 6 shows that in the presence of various concentrations of SDS, binding of S. mutans to Sephadex® G-25 is not affected. This demonstrates that the components of toothpaste may not interfere with this reaction.

Effect of pH on Adherence

Under normal conditions in the oral cavity the pH can vary from 3-7.5 depending on multiple conditions, for example, recently ingested food (e.g. sugar) drops the pH precipitously. Therefore, a wide pH range will be evaluated to determine if these alterations in pH would affect S. mutans ability to bind to Sephadex®. For these experiments, overnight cultures are diluted into fresh THB or the artificial saliva with pH ranging from 3.0 to 8.0, grown to mid-log phase and incubated for 1 minute with the optimal Sephadex® slurry. This will demonstrate if the binding reaction can occur under the various conditions encountered in the oral cavity.

In the oral cavity S. mutans binds to the surface of the tooth. Human tooth enamel is comprised of carbonated and fluoridated hydroxyapatite minerals. What extent S. mutans will bind Sephadex® in the presence of hydroxyapatite determines how much Sephadex® is required for therapeutic uses. Ceramic and crystalline hydroxyapatite are commercially available (BioRad, Hercules, Calif.) and experiments with our column assay similar to the initial experiments with Sephadex®, to confirm that S. mutans will preferentially bind Sephadex® in the presence of hydroxyapatite and at each of the different growth phases of S. mutans including planktonic and biofilms cultures.

Competition experiments to determine the extent of S. mutans ability to compete with hydroxyapatite were performed using hydroxyapatite (HA) rods to mimic the surface of teeth. The rods were cut into discs and used in the presence of Sephadex®. The use of discs allows for the two compounds to be separated from each other for comparison.

To quantitate the amount of bacteria binding to the Sephadex® and HA discs the column assays are not applicable for this experiment. To quantitate the amount of bacteria bound to each compound, reporter strains of S. mutans UA140 constitutively expressing the luciferase gene from the lactate dehydrogenase, ldh, promoter was used [UA140::φ(ldhp-luc)]. This strain was grown as planktonic and biofilm cultures to various growth phases. Both Sephadex® and a HA disc were incubated together and separately with 0.5 ml of culture at room temperature for one minute. The compounds are pelleted by centrifugation and the unbound S. mutans are removed. The HA disc was removed with sterilized tweezers and both compounds were resuspended in 0.5 ml of either THB or artificial saliva and used for luciferase reporter assays that have been used in our laboratory previously. The percentage of bacteria that were able to bind to either the Sephadex® or HA disc is calculated by comparing to measurements of the cultures without incubation with either compound.

The amount of Sephadex® added to the reactions can be altered to determine how much Sephadex® is required to out compete the binding of S. mutans to the HA. This amount of Sephadex® will be utilized for the remainder of the experiments and the amount of bacteria that can be depleted from the oral cavity will be calculated.

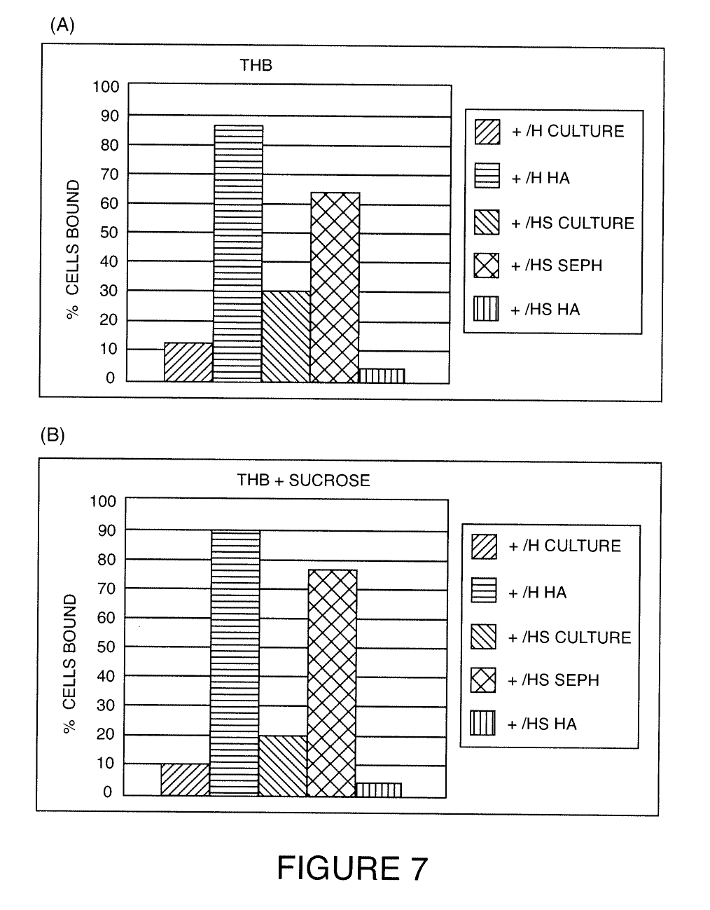

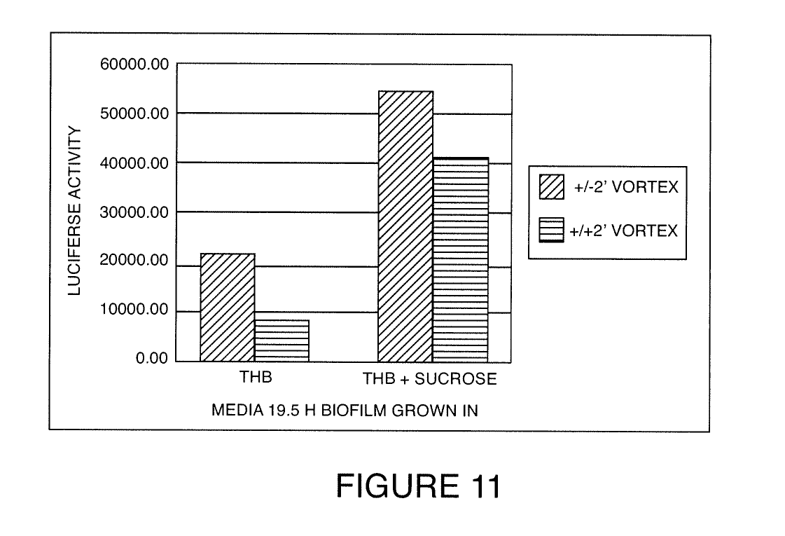

FIG. 7 shows the result of competition assay in which planktonic S. mutans cultures were incubated in the presence of both HA disc and Sephadex® for a short period of time (5 minutes). S. mutans appears to preferentially bind to the Sephadex® over the HA discs. FIG. 11 shows the results of a S. mutans biofilm developed on the HA disc and then incubated with Sephadex® to pull the S. mutans out of the biofilm.

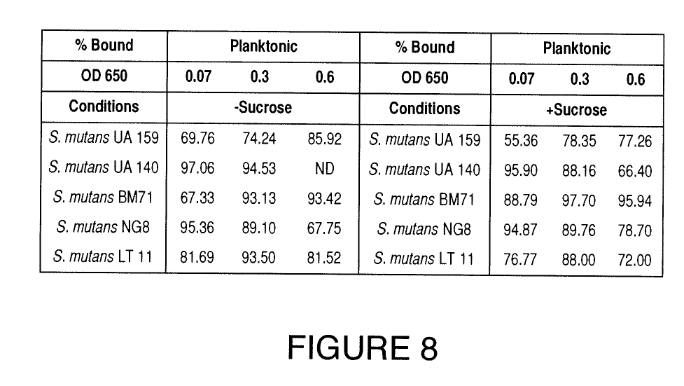

FIG. 8 shows binding of S. mutans in planktonic culture.

Testing for Binding of Wide-Spectrum Oral Bacteria to Sephadex®

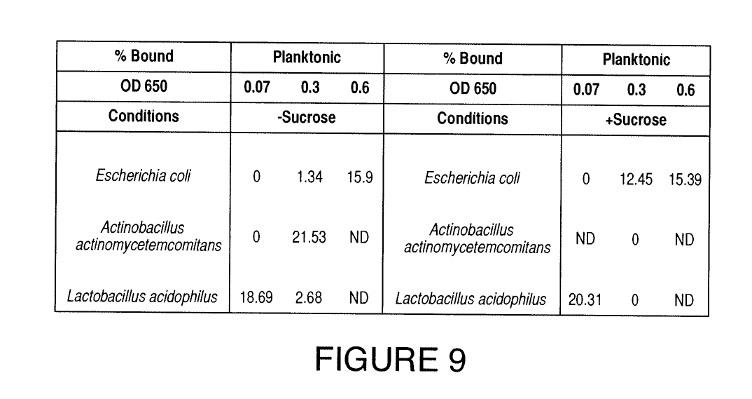

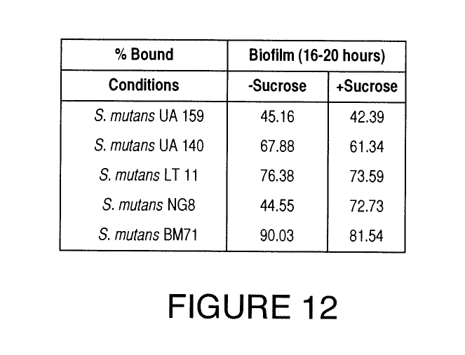

An extensive strain collection of oral bacteria is known. One may investigate the binding of different S. mutans strains (FIG. 12) and a broader range of oral bacteria to confirm binding specificity for caries-causing bacteria (FIG. 9).

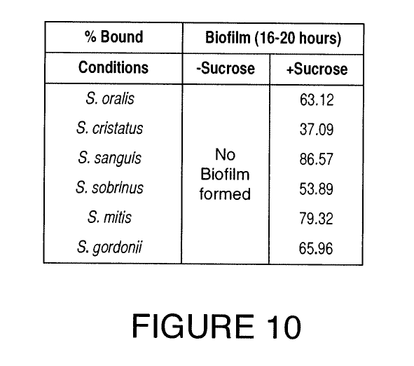

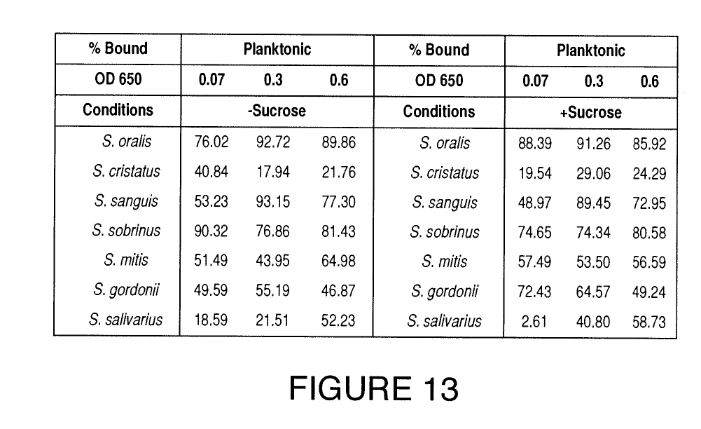

FIG. 10 shows binding of various streptococci in biofilm culture. FIG. 13 shows binding of various streptococci in planktonic culture.

Examples of the other possible oral bacteria that may be tested include: early colonizers: Actinomyces israelii, Actinomyces naeslundii, Capnocytophaga gingivalis, Capnocytophaga ochracea, Capnocytophaga sputigena, Fusobacterium nucleatum, Haemophilus parainfluenzae, Prevotella denticola, prevotella loescheii, Propionibacterium acnes, Streptococcus gordonii, Streptococcus mitis, Streptococcus oralis, Streptococcus sanguis and Veillonella atypical late colonizers: Actinomyces actinomycetemcomitans, Eubacterium sp, Porphyromonas gingivalis, Prevotella intermedia and Lactobacillus.

Binding of S. mutans in Mature Biofilm to Sephadex®

Experiments show that S. mutans is capable of binding to Sephadex® as planktonic cells. Another stage of development of S. mutans is a mature biofilm where the cells are completely embedded in the EPS layer. Mature biofilms of S. mutans UA140::φ(ldhp-luc) are grown on HA discs for various lengths of time (e.g. 8-48 hour biofilms). The Sephadex® slurry are then added to the biofilms and incubated for 1-30 minutes and the amount of bacteria depleted from the biofilm are quantified by the luciferase reporter assay and compared to biofilms that are not incubated with the Sephadex® slurry. This demonstrates whether Sephadex®, when added to chewing gum as a delivery system, is able to deplete S. mutans from the biofilm in the oral cavity.

FIG. 12 shows the result of different S. mutans strains binding to Sephadex® in biofilm.

Label Various Oral Bacteria Strains for Broad-Spectrum Competition Test

As previously mentioned, biofilms are a complex community made up of a variety of bacteria. A competition experiment will involve planktonic cultures or biofilms grown with a mixed culture of S. mutans and one other oral bacterial strain (e.g. S. mutans and S. gordonii). The two different bacteria in the mixed cultures are distinguishable by labeling each with a different radioisotope. To label the bacteria with a radioisotope, the bacteria will be cultured in THB containing either 0.5 mCi of [³H]-thymidine or [¹⁴C]-thymidine (Perkin Elmer) and incubated at 37° C. Once the cells reach the desired growth phase the cells will then be harvested by centrifugation and excess radioactivity will be removed. For the competition experiments the labeled cells are mixed at various ratios and allowed to grow as a biofilm or planktonic cells. At the appropriate growth phase the Sephadex® slurry will be added and incubated at room temperature. After the unbound cells are removed, the amount of bound and unbound bacteria will be measured with a scintillation counter capable of detecting [₃H] and [¹⁴C]. This will determine if the binding of S. mutans to the Sephadex® disrupts other bound bacteria.

The most appropriate method to use Sephadex® in an oral health care line to remove S. mutans from the oral cavity may be determined. To facilitate the selection of therapeutic modality one may first determine which of the various components of toothpaste, mouthwashes or chewing gum are conducive for S. mutans binding to Sephadex®. Studies with SDS demonstrated that there was no effect observed with the binding.

All of the major components of toothpaste, mouthwashes and chewing gum individually (e.g. various sugars, alcohol, sodium fluoride, etc) and as a whole product in the column binding assay may be tested by resuspending the ingredient in the artificial saliva before the addition of the cells and comparing the amount of binding with and without the various components.

While the experiments above speak to the tolerance of S. mutans binding to Sephadex®, here it may be advantageous to potentiate binding to Sephadex®. As previously mentioned, S. mutans exhibit rapid aggregation of cells under stress conditions, known as the ddag phenomenon and GpbC mediated this binding to dextran. Therefore, the basis of the S. mutans binding to Sephadex®, cross-linked dextran, also likely involves GbpC.

Sato and colleagues have demonstrated the induction of the ddag phenomenon by growing cells in the presence of various stresses (antibiotics, amino acid analogues, ethanol and xylitol). Thus, one may choose substances to be added with the Sephadex® that are otherwise categorized as ‘generally regarded as safe (GRAS)’, that could potentiate binding to Sephadex® by stressing S. mutans. For our purposes we want to induce the ddag phenomenon by adding these compounds individually to the saliva before S. mutans cultures are resuspended in the saliva and incubated with the Sephadex® slurry for 1-30 minutes for the column based binding assay to determine if enhanced binding to Sephadex® is observed. As a control S. mutans strain GS5 will be used because the strain does not possesses a mutant gbpC gene, therefore, does not have the ddag phenomenon. If increased binding is observed the appropriate ingredient will be added to the product so the largest amount of bacteria will be removed from the mouth with every use of the product containing Sephadex®.

The foregoing provides the basis of a new noninvasive method for specifically removing the caries-causing bacteria, S. mutans from the oral cavity. These studies demonstrated that a small “pea-sized” portion of Sephadex® was required to remove all of the S. mutans in an individual with severe caries and that other oral bacteria were not disturbed. The use of Sephadex® in toothpaste, mouthwash or chewing gum will be inexpensive and simple. In addition, Sephadex® is non-toxic and tasteless.

Pre-clinical experiments may be conducted to demonstrate the binding efficiency and selectivity of Sephadex® for S. mutans in freshly harvested human saliva, test competitive binding with Sephadex® and extracted teeth (with and without saliva), degradation of Sephadex® in human saliva, and our initial formulations of each of the oral hygiene products (toothpaste, mouthwash or chewing gum).

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A method for removing caries-causing agents from a site, comprising:
applying a glucosidic polymer-based caries-causing agent removal device to the oral cavity so as to cause attachment of the caries-causing agents to the device, wherein the; and
removing the device from the oral cavity, thereby, removing the caries-causing agents from the oral cavity.

2. The method of claim 1, wherein said site is the oral cavity of a subject.

3. The method of claim 1, wherein said caries-causing agent is a cariogenic streptococcal species.

4. The method of claim 1, wherein said caries-causing agent is S. mutans, S. sobrinus, S. oralis, S. sanguis, S. mitis, S. salivarius, S. cristatus or combinations thereof.

5. The method of claim 1, wherein said glucosidic polymer-based device comprises an insoluble polymer having an α-1,3 cross-linked, an α-1,6 cross-link glycosidic bond or combinations thereof.

6. The method of claim 1, wherein said glucosidic polymer-based device comprise an insoluble cross-linked dextran.

7. The method of claim 1, wherein said glucosidic-based device comprises Sephadex®, sepharose, or a mimetics thereof.

8. The method of claim 1, wherein said removal of the device is by swallowing.

9. The method of claim 1, wherein said removal of the device is by disposing the device at a disposal site external to the subject.

10. The method of claim 1, wherein said applying is by way of repeatedly contacting the device with the caries-causing agent for a predetermined period of time.

11. The method of claim 1, wherein said glucosidic polymer-based caries-causing agent removal device is in the form of a candy, chewing gum, toothpaste, or mouthwash.

12. The method of claim 1, wherein said applying step is performed within a predetermined amount of time after the site is exposed to caries-causing agent or nutrients that foster the growth of caries-causing agents.

13. The method of claim 1, further comprising applying a dextran-dependent aggregation inducer to the site.

14. The method of claim 1, wherein the device further includes a dextran-dependent aggregation inducer.

15. The method of claim 13, wherein the dextran-dependent aggregation inducer is one selected from an antibiotics, an amino acid analog, ethanol, xylitol, elevated temperatures, or a combination thereof.

16. A caries-causing agent removal device for removing caries-causing agents from a site, comprising:
a glucosidic polymer-based body
wherein the caries-causing agents form selective and preferential attachment to the device.

17. The device of claim 16, wherein the glucosidic polymer-based body is comprised of an insoluble cross-linked dextran.

18. The device of claim 16, wherein the glucosidic polymer-based body is comprised of Sephadex®, or a mimetics thereof.

19. The device of claim 16, wherein the device is in the form of candy, chewing gum, toothpaste, mouthwash, or combination thereof.

20. The device of claim 16, wherein the device is capable of selectively removing S. mutans, S. sobrinus, S. oralis, S. sanguis, S. mitis, S. salivarius, S. cristatus, or combinations thereof from the site.

21. A dental cleaning apparatus, comprising:
a main body; and
a cleaning surface for contacting and selectively attaching caries-causing agents from a site,
wherein the surface is comprised of glucosidic polymer-based material and is connected to the main body.

22. The dental cleaning apparatus of claim 21, wherein the surface is comprised of an insoluble cross-linked dextran.

23. The dental cleaning apparatus of claim 21, wherein the surface is comprised of Sephadex®, sepharose, or a mimetics thereof.

24. The dental cleaning apparatus of claim 21, wherein the surface is capable of selectively removing S. mutans, S. sobrinus, S. oralis, S. sanguis, S. mitis, S. salivarius, S. cristatus, or combinations thereof.

25. The dental cleaning apparatus of claim 21, further comprises a mechanical applicator element for generating an oscillating movement in the cleaning surface, wherein the applicator element is connected to the cleaning surface and wherein during operation the cleaning surface is made to contact the site.

26. The dental cleaning apparatus of claim 21, wherein the surface is detachable from the main body for replacement.
